(19) United States
(12) Patent Application Publication
(10) Pub. No.: US 2013/0072990 A1
(43) Pub. Date: Mar. 21, 2013

(54) REVERSE THREAD BONE SCREW

(71) Applicant: Peter Melott Simonson, Longboat Key, FL (US)

(72) Inventor: Peter Melott Simonson, Longboat Key, FL (US)

(21) Appl. No.: 13/622,334

(22) Filed: Sep. 18, 2012

Related U.S. Application Data
(60) Provisional application No. 61/536,501, filed on Sep. 19, 2011.

(51) Int. Cl. A61B 17/86

(52) U.S. Cl. 606/301

(57) ABSTRACT

A left-handed bone screw for use in orthopedic spinal surgical procedures. The left-handed bone screw reverses the pitch angle of a conventional right-handed bone screw. In reversing the thread, the present invention permits the left-handed screw to reuse a previously tapped cortical and cancellous bone hole.

### CROSS-REFERENCE TO RELATED APPLICATIONS

This application claims priority to U.S. Provisional Application No. 61/536,501, filed on Sep. 19, 2011, the disclosure of which is incorporated herein by reference in its entirety for all purposes.

### FIELD OF INVENTION

The present invention relates to bone screws used in orthopedic spinal surgical procedures. Specifically, the invention is directed to a left-handed bone screw.

### BACKGROUND OF THE INVENTION

Several techniques and systems have been developed for correcting and stabilizing the spine and facilitating spinal fusion. Over the years, spinal and orthopedic implants have evolved toward progressively stronger and stiffer devices, since it is presumed that increased construct rigidity optimizes the bone fusion and provides more rapid and robust healing. The most widely used systems use a bendable rod that is placed longitudinally along the length of the spine. In such a procedure, a rod is attached to various vertebrae along the length of the spinal column by a number of bone anchor assemblies. A bone anchor element may be a hook that engages the vertebra laminae or a bone screw threaded into the vertebral bone.

In present day bone screw assemblies, rods are typically situated on opposite sides of the spine or spinous processes. Numerous bone screws are screwed into the pedicles of the vertebral bodies. Rods are then affixed to these bone screws through various connectors so corrective and stabilizing forces are applied to the spine. When stabilized, the vertebra is decortified where the outer cortical bone is removed to provide a foundation for bone grafts. Over time, these bone grafts fuse the damaged vertebrae together.

Bone screws are well known in the art. The threads, for example, of a bone screw anchor it to the bone and keep the screw from being axially pulled out of the bone. Such threads also cut a helical path into the bone as the screw rotates into the bone.

As shown in FIG. 1, a right-handed bone screw 2 has male threads 4, while its matching nut or substrate possess corresponding female threads. Almost all threads are so oriented that when a matching nut is turned clockwise, the nut moves down the screw. This is known as right-handedness. When turned counter-clockwise, the nut moves up the screw; this is known as left-handedness. Right-handedness is the default for bone screw threads. Returning to FIG. 1, a screw consists of a major diameter 6 and a minor diameter 8. The major diameter 6 is the large diameter of the thread. The minor diameter 8 is the small diameter of the thread. Pitch 12 is the distance from the crest 14 of one thread to the next. The root 16 of the male thread 4 touches the inside diameter 8. Screw threads are almost never made perfectly sharp but instead are truncated. This truncation is known as the thread depth 18. Threads are strongest when they are truncated about 60% so that the increased material thickness is achieved at the crest 14 of the screw. The angle of the cross-sectional shape is called the pitch angle 20.

There are many aspects of thread shape in the art. The thread can have a sharp or blunt apex. The pitch of the thread can be varied. The height of the thread can be deep or shallow. The thickness of the thread can change. In all screws, however, threads have a superior surface facing the screw head and an inferior surface facing the screw tip. The engagement of the superior surface with the bone provides resistance to screw pull-out. The screw thread can be further modified by varying the cross-sectional shape of the thread from the tip to the head of a screw. In most screws, the thickness of the thread near the tip has a narrow cross-section to cut into the bone as the screw rotates into the bone. The thread and core become thicker toward the head of the screw to increase thread strength and to displace bone matter downward against the superior thread surface. This is particularly advantageous in bones because of the hard cortical bone shell. The cortical bone is harder and more compact than the spongy cancellous bone in the bone center. The cortical bone provides the bulk of the bone's resistance to screw pull-out forces (axial load forces). The thread and core near the screw head engages the cortical bone and, thus, carry much of axial load on the screw.

A common problem among all bone screw assemblies is bone screw pull-out. When bone screws are weakened or begin to pullout, bone anchor or screw assemblies may slip. When the bone is strong and healthy, the initial fixation of the spinal and orthopedic screw is usually excellent, with more than adequate pull-out strength. With dense, sclerotic or osteoporotic bone, micro-motions resulting from the normal range of motion within the skeletal system may lead to a progressive degradation from the initially implanted state. In cases where the bone fails to heal, these micro-motions persist and cause the metallic screw to oscillate within the softer cancellous bone. When subjected to persistent toggling with the modulus mismatch of the metal to cortico-cancellous bone, the bone screw becomes loose.

During bone screw pullout, dislodgement and breakage occur because of extreme load, shear, stress and/or torsion. When bone screw failure occurs, it not only weakens the mechanical strength of the bone screw assembly but also lowers the biological potential for bone healing. When bone screws loosen, removal may be necessary because the earlier threads made in the bone by the bone screw are no longer usable. Given the importance of screw threads, it is surprising that there is no or little reference known on the use of right-handedness or left-handedness bone screws.

In summary, there is a need in the industry for improvements in bone screw and bone screw thread design. The present invention describes such an improvement.

### BRIEF SUMMARY OF THE INVENTIONS

The present invention provides an improvement to bone screws, such as vertebral pedicle bone screws, used in orthopedic spinal surgical procedures. Specifically, the present invention reverses the pitch angle to left-handedness from right-handedness.

When bone screw failure necessitates bone screw removal, the reverse thread of the present invention permits the screw to return through the previously tapped cortical and cancellous bone substrate and, more importantly, cuts and taps through the new bone providing a new bone screw with added strength and integrity.

### BRIEF DESCRIPTION OF THE DRAWINGS

FIG. 1 shows a side view of a prior art right-handed screw.

FIG. 2 is a side view of left-handed screw of the present invention.

FIG. 3 shows a close-up side view of the left-handed bone screw.

FIG. 4 is an example of the reverse thread of the present invention threaded through new bone substrate that was tapped earlier by a right-handed screw.

### DETAILED DESCRIPTION OF THE INVENTION

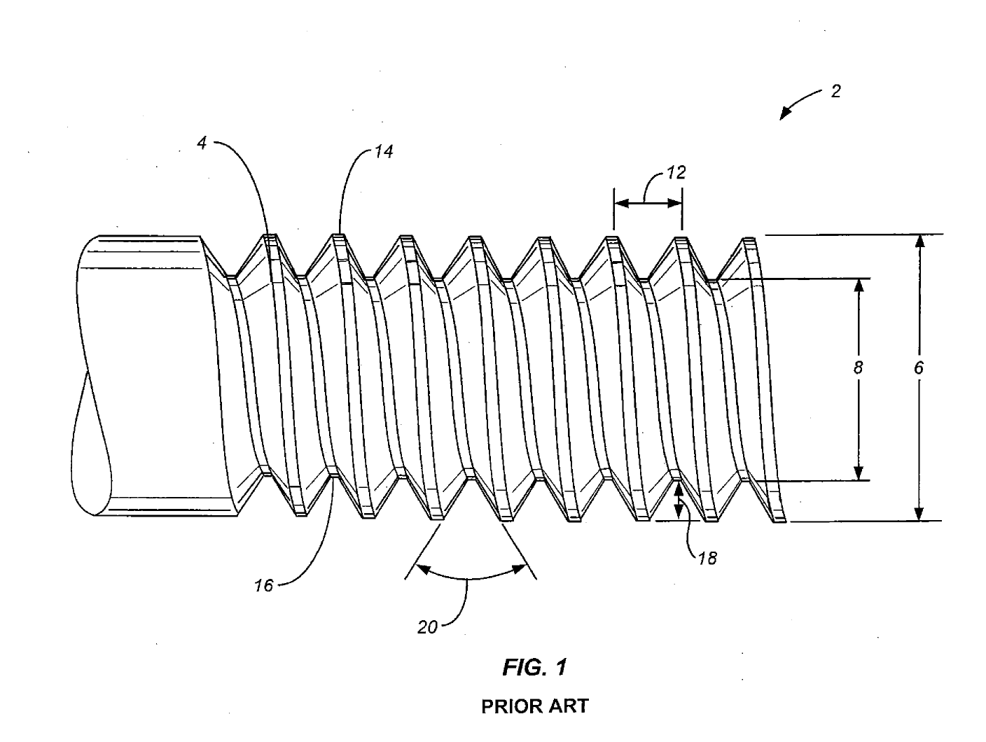

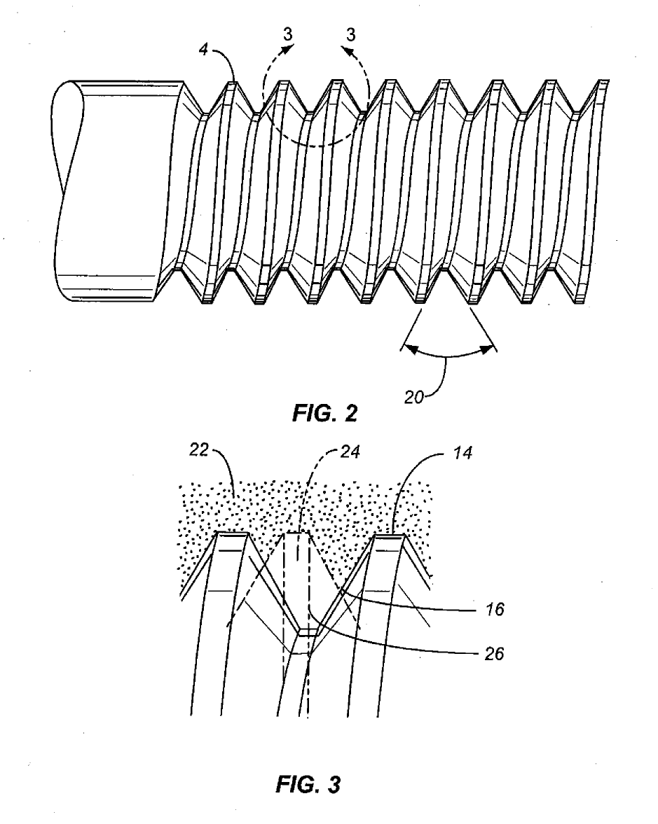

The present invention is shown in FIGS. 2, 3 and 4. The male threads 4 shown in FIGS. 1 & 2 are shifted from a negative slope on the vertical (x-y axis) in FIG. 1 to a positive slope on the vertical (x-y-axis) in FIG. 2. In other words, the pitch angle 20 of the male threads 4 in FIG. 1 is simply reversed to left-handedness from right-handedness in FIG. 2.

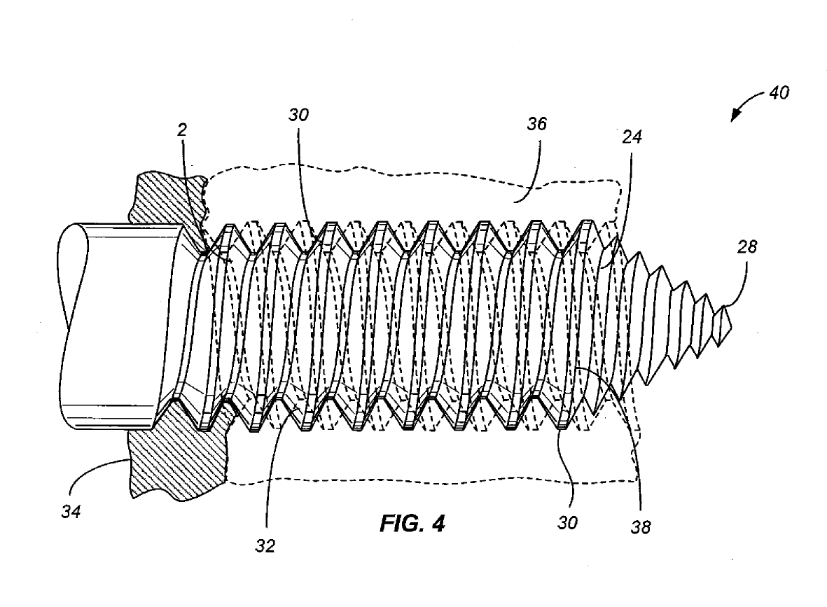

When the male threads 4 are reversed, the pitch angle 20 is shifted either one thread up or down. As the male threads 4 are reversed, the male threads 4 are shifted into new bone substrate 22. This is illustrated in FIG. 3. When the thread is reversed, the crest 14 and, perhaps, a portion of the root 16 is now surrounded by new bone substrate 22. When the male threads 4 are reversed, a void 24 left over from the previous position of the female thread 26 is also created.

As shown in FIG. 4, the tip 28 of the reverse thread 30 passes through the hole 32 left by the previous right-handed bone screw 2. The tip 28 passes through the cortical bone 34 without cracking or damaging the bone. The reverse thread 30 of the present invention displaces the cancellous bone 36 by cutting and pushing new cancellous bone 36 downward. The leading edge 38 of the left-handed bone screw 40 cuts a new passageway in the bone for the reverse thread 30.

As the left-handed bone screw 40 is rotated downward into the bone, the core thickness of the left-handed bone screw 40 increases. This core continually pushed cancellous bone 36 radiallly outward and, thus, slightly compresses the cancellous bone 36 in between the reverse threads 30. The cancellous bone 36 immediately below the inferior surface is displaced downward against the superior reverse thread 30 compacting the cancellous bone 36 between the reverse threads 30 and against the left-handed bone screw 40. As it does so, it fills in the void 24 left previously by the right-handed bone screw 2. The new cancellous bone 36 in the void 24 now has an opportunity to heal and strengthen the void 24. New cancellous bone 36 may even replace and fill in the void 24 left in the cortical bone 34, thereby, also strengthening it. This compression of the cancellous bone 36 between the reverse threads 30 also orients the bone to oppose screw pull-out. The compaction and orientation of the new bone improves the support provided by the cancellous bone 36, thereby increasing the pull-out resistance of the reverse thread 30 and the left-handed bone screw 40.

The left-handed bone screw 40 and its reverse thread 30 described herein can be customized for particular bone applications. For example, the taper, diameter and cross-sectional shape of the bone screw can be modified on the left-handed bone screw 40 just as the right-handed bone screw 2. The reverse thread 30 may also possess different pitch, height and thickness as other right-handed bone screws 2.

With the present invention, a surgeon can now use the previous right-handed bone screw hole with a left-handed screw having similar or the same size bone screw and similar pitch. The present invention avoids the use of a larger size right-handed bone screw that, in most cases, will further crack and damage the bone. The present left-handed invention also provides the new bone screw with increased strength and integrity. Furthermore, it increases the mechanical strength of the bone screw assembly and may also increase the biological potential for bone healing.

In the foregoing specification, the invention has been described with reference to specific preferred embodiments and methods. It will, however, be evident to those of skill in the art that various modifications and changes may be made without departing from the broader spirit and scope of the invention. Accordingly, the specification and drawings are to be regarded in an illustrative, rather than restrictive, sense; the invention being limited only by the appended claims.

What is claimed is:

1. A bone screw having left-handed threads.

2. The bone screw of claim 1 wherein said screw has a narrower diameter tip and a wider diameter head section.

3. The bone screw of claim 1 wherein said bone screw is a vertebral pedicle bone screw.

4. A method of replacing a right-handed bone screw comprising the step of:
removing said right-handed bone screw from a vertebrae to create a cavity in said vertebrae;
selecting a left-handed bone screw of comparable length and diameter to said right-handed bone screw;
inserting said left-handed bone screw into the vertebrae cavity formed by the removed right-handed bone screw.
